(19) United States
(12) Patent Application Publication
(10) Pub. No.: US 2025/0201361 A1
(43) Pub. Date: Jun. 19, 2025

(54) MEDICAL INFORMATION PROCESSING DEVICE, MEDICAL INFORMATION PROCESSING METHOD, AND NON-TRANSITORY COMPUTER READABLE MEDIUM

(71) Applicant: CANON MEDICAL SYSTEMS CORPORATION, Otawara-shi (JP)

(72) Inventors: Junichi TAKAZAWA, Nasushiobara (JP); Hiroki SAITO, Oyama (JP); Kohei SHINOHARA, Nasushiobara (JP); Nobuki MOROOKA, Nasushiobara (JP); Hideyuki SUZUKI, Nasushiobara (JP)

(73) Assignee: CANON MEDICAL SYSTEMS CORPORATION, Otawara-shi (JP)

(21) Appl. No.: 18/983,668

(22) Filed: Dec. 17, 2024

(30) Foreign Application Priority Data
Dec. 19, 2023 (JP) 2023-213454
Dec. 16, 2024 (JP) 2024-220051

(51) Int. Cl. G16H 10/60; G16H 20/10; G16H 30/20

(52) U.S. Cl. CPC G16H 10/60; G16H 20/10; G16H 30/20

(57) ABSTRACT

A medical information processing device according to an embodiment includes a processor. The processor acquires adverse event information that indicates an adverse event occurred in a patient. The processor extracts administration information that indicates a drug administered to the patient. From the administration information, the processor identifies a related drug related to the occurrence of the adverse event. The processor identifies related image information on the related drug, if there is a related drug. The processor displays output information that includes the adverse event information, the administration information, drug information on the drug, and the related image information.

### CROSS-REFERENCE TO RELATED APPLICATIONS

This application is based upon and claims the benefit of priority from Japanese Patent Application No. 2023-213454, filed on Dec. 19, 2023; and Japanese Patent Application No. 2024-220051, filed on Dec. 16, 2024; the entire contents of which are incorporated herein by reference.

### FIELD

Embodiments described herein relate generally to a medical information processing device, a medical information processing method, and a non-transitory computer readable medium.

### BACKGROUND

For example, when drugs such as cardiotoxic anticancer drugs are to be administered to a patient, physicians are recommended to perform regular cardiac function tests (cardiac function monitoring) before the treatment, after the treatment is started, and during the treatment. For example, if a patient develops cardiotoxicity, physicians check various information such as anticancer drugs administered to the patient, adverse events including cardiac disorder or the like due to the administration of anticancer drugs, and captured images that indicate the results of cardiac function tests, to diagnose the patient. However, there is room for further improvement as the information described above are not associated with each other, and physicians are finding it difficult to diagnose.

### BRIEF DESCRIPTION OF THE DRAWINGS

FIG. 1 is a block diagram illustrating a medical information processing device according to an embodiment;

FIG. 2 is a table illustrating an example of adverse event information according to the embodiment;

FIG. 3 is a table illustrating an example of administration information according to the embodiment;

FIG. 4 is a table illustrating an example of detailed drug information according to the embodiment;

FIG. 5 is a table illustrating an example of image information according to the embodiment;

FIG. 6 is a diagram for explaining an example of output information according to the embodiment;

FIG. 7 is a flowchart illustrating an example of a process of a medical information processing device according to the embodiment;

FIG. 8 is a block diagram illustrating a medical information processing device according to a first modification;

FIG. 9 is a diagram for explaining an example of output information according to the first modification;

FIG. 10 is a flowchart illustrating an example of a process of the medical information processing device according to the first modification;

FIG. 11 is a diagram for explaining an example of output information according to a second modification; and

FIG. 12 is a flowchart illustrating an example of a process of the medical information processing device according to the second modification.

### DETAILED DESCRIPTION

A medical information processing device according to an embodiment includes a processor. The processor acquires adverse event information that indicates an adverse event occurred in a patient. The processor extracts administration information that indicates a drug administered to the patient. From the administration information, the processor identifies a related drug related to the occurrence of the adverse event. The processor identifies related image information on the related drug, if there is a related drug. The processor displays output information that includes the adverse event information, the administration information, drug information on the drug, and the related image information.

Hereinafter, embodiments of a medical information processing device, a medical information processing method, and a non-transitory computer readable medium will be described in detail with reference to the accompanying drawings. However, the medical information processing device, the medical information processing method, and the non-transitory computer readable medium according to the present application are not limited to the embodiments described below.

### Embodiment

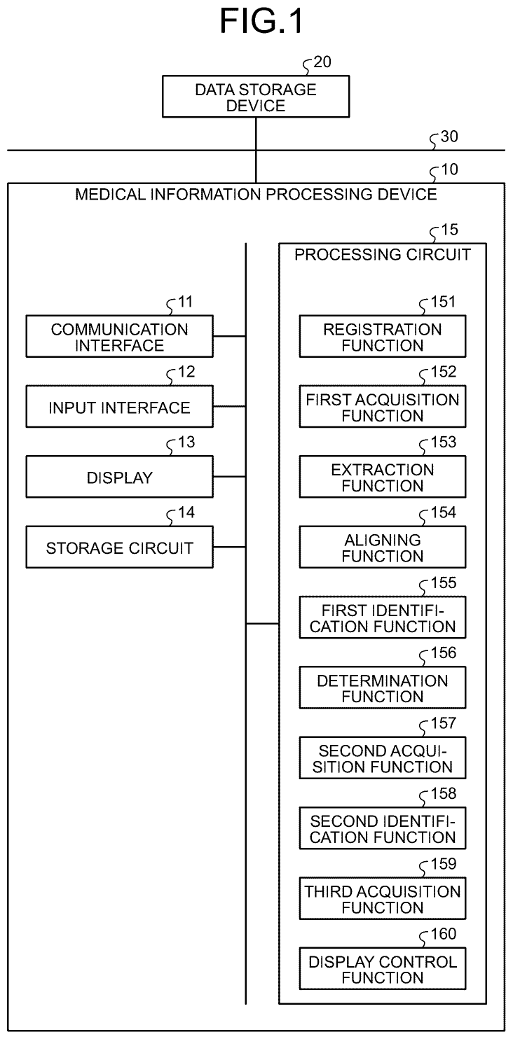

FIG. 1 is a block diagram illustrating a medical information processing device 10 according to an embodiment. For example, as illustrated in FIG. 1, the medical information processing device 10 according to the present embodiment is communicatively connected to a data storage device 20 via a network 30. In this example, the network 30 includes an in-house local area network (LAN) installed in a hospital or a wide area network (WAN), for example. Only the medical information processing device 10 and the data storage device 20 are connected to the network 30 illustrated in FIG. 1. However, in practice, various other devices and systems are connected to the network 30.

The data storage device 20 stores information such as medical information (clinical data) of patients, and various guidelines. Specifically, the data storage device 20 receives data on medical information (raw data, a medical image, analysis results, reports, and the like) from various devices and systems connected to the network 30, and stores the received data on medical information in a storage circuit within the device.

Moreover, the data storage device 20 stores the data on the information in the storage circuit within the device, according to the operation for storing information such as guidelines and documents. For example, the data storage device 20 is implemented by a picture archiving and communication system (PACS), a hospital information system (HIS), a radiology information system (RIS), and the like.

For example, the medical information processing device 10 is a device operated by a physician working in a hospital, and executes various processes for improving the diagnostic efficiency of physicians, compared to the conventional one. For example, if a patient develops cardiotoxicity, physicians check various information such as anticancer drugs administered to the patient, adverse events including cardiac disorder due to the administration of anticancer drugs, and captured images that indicate the results of cardiac function tests, to diagnose the patient. The medical information processing device 10 associates the information described above, and allows the physicians to check the associated information and diagnose the patient. Hence, it is possible to improve the diagnostic efficiency of physicians, compared to the conventional one. For example, the medical information processing device 10 is implemented by a computer device such as a personal computer (PC), a workstation, and a server.

As illustrated in FIG. 1, the medical information processing device 10 according to the present embodiment includes a communication interface 11, an input interface 12, a display 13, a storage circuit 14, and a processing circuit 15.

The communication interface 11 controls the transmission and communication of various data transmitted and received between the medical information processing device 10 and the devices connected via the network 30. Specifically, the communication interface 11 is connected to the processing circuit 15, and transmits the data received from the devices on the network 30 to the processing circuit 15, or transmits the data received from the processing circuit 15 to the devices on the network 30. For example, the communication interface 11 is implemented by a network card, a network adapter, a network interface controller (NIC), and the like.

The input interface 12 receives an input operation of various instructions and information from an operator. Specifically, the input interface 12 is connected to the processing circuit 15, and converts the input operation received from the operator into an electrical signal, and transmits the converted electrical signal to the processing circuit 15. For example, the input interface 12 is implemented using a trackball, a switch button, a mouse, a keyboard, a touch pad with which input operations are performed by touching an operation surface, a touch screen in which a display screen and a touch pad are integrated, a non-contact input interface using an optical sensor, a voice input interface, and the like.

In the present specification, the input interface 12 is not limited to one having physical operation parts such as a mouse and a keyboard. For example, an electrical signal processing circuit that receives an electrical signal corresponding to an input operation from an external input device, which is provided separately from the device, and that transmits the electrical signal to the control circuit, is also an example of the input interface 12.

The display 13 displays various types of information and data. Specifically, the display 13 is connected to the processing circuit 15, and displays various types of information and data received from the processing circuit 15. For example, the display 13 is implemented using a liquid crystal display, a cathode ray tube (CRT) display, a touch panel, and the like. The display 13 is an example of a display unit.

The storage circuit 14 stores various data and computer programs. Specifically, the storage circuit 14 is connected to the processing circuit 15, and stores data received from the processing circuit 15, or reads the stored data and transmits the read data to the processing circuit 15. For example, the storage circuit 14 is implemented using a semiconductor memory element such as a read only memory (ROM), a random access memory (RAM), and a flash memory, a hard disk, an optical disc, or the like. The storage circuit 14 may be implemented using a cloud computer connected to the medical information processing device 10 via the network 30.

The processing circuit 15 controls the entire medical information processing device 10. Specifically, the processing circuit 15 executes various processes for improving the diagnostic efficiency of physicians. For example, the processing circuit 15 controls the exchange of data with a device on the network 30, the storage of data in the storage circuit 14, and various processes using data.

For example, as illustrated in FIG. 1, in the present embodiment, the processing circuit 15 of the medical information processing device 10 executes a registration function 151, a first acquisition function 152, an extraction function 153, an aligning function 154, a first identification function 155, a determination function 156, a second acquisition function 157, a second identification function 158, a third acquisition function 159, and a display control function 160.

The registration function 151 registers adverse event information. The registration function 151 is an example of a registration unit. Specifically, by cooperatively working with the input interface 12 operated by an operator, the registration function 151 registers adverse event information that indicates the adverse events occurred in a patient, in the storage circuit 14. For example, the adverse event information includes symptoms of cardiotoxic adverse events caused by anticancer drugs.

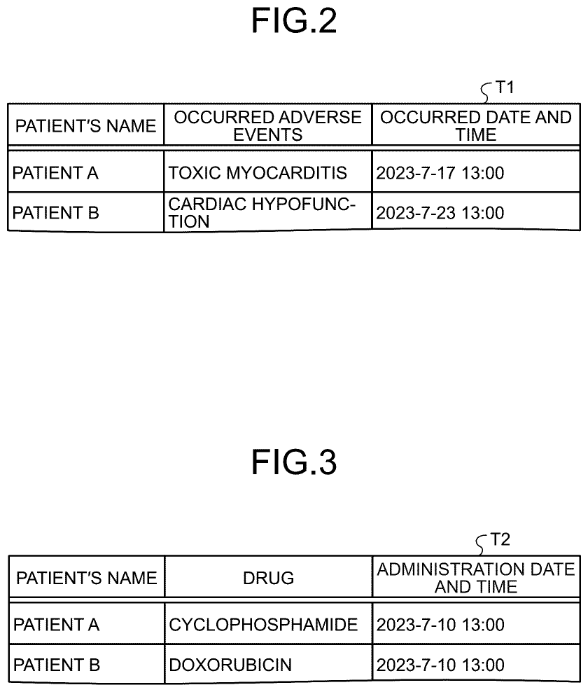

The adverse event information registered in the storage circuit 14 by the registration function 151 will now be described with reference to FIG. 2. FIG. 2 is a table illustrating an example of adverse event information according to the embodiment. As illustrated in FIG. 2, adverse event information T1 is information that includes patient information including the patient's name, occurred adverse events, and occurred date and time information including the date and time when the patient has developed cardiotoxicity.

The patient information includes the patient's name and an identifier for identifying the patient. The occurred adverse events are not limited to toxic myocarditis and cardiac hypofunction illustrated in FIG. 2, but also include symptoms of cardiotoxic adverse events caused by anticancer drugs. For example, such adverse events include left ventricular dysfunction, myocardial ischemia, arrhythmia, toxic myocarditis, and the like.

However, the adverse event information T1 is not limited thereto, and may also include severity information that indicates the severity of the adverse event, for example. For example, the severity information is a criterion defined in the Common Terminology Criteria for Adverse Events (CTCAE). However, the severity information is not limited thereto. The adverse event information T1 may also include registration date and time information that includes the date and time when the registration function 151 has registered the adverse event information in the storage circuit 14.

Returning to FIG. 1, the first acquisition function 152 acquires adverse event information. The first acquisition function 152 is an example of a first acquisition unit. Specifically, the first acquisition function 152 acquires adverse event information that indicates the adverse event occurred in a patient, that is registered by the registration function 151, from the storage circuit 14.

The extraction function 153 extracts administration information. The extraction function 153 is an example of an extraction unit. Specifically, the extraction function 153 extracts administration information that indicates the drugs administered to the patient, from the storage circuit 14, on the basis of the patient information included in the adverse event information acquired by the first acquisition function 152.

The administration information extracted from the storage circuit 14 by the extraction function 153 will now be described with reference to FIG. 3. FIG. 3 is a table illustrating an example of administration information according to the embodiment. As illustrated in FIG. 3, administration information T2 is information that includes patient information including the patient's name, drugs administered to the patient included in drug information, and administration date and time information including the administration date and time when the drug is administered to the patient. The drug information administered to the patient is not limited to cyclophosphamide and doxorubicin illustrated in FIG. 3.

Returning to FIG. 1, the aligning function 154 aligns anticipated period information. The aligning function 154 is an example of an aligning unit. Specifically, on the basis of the administration information extracted by the extraction function 153, the aligning function 154 aligns the anticipated period information linked to the drug included in the drug information, from the detailed drug information stored in the storage circuit 14.

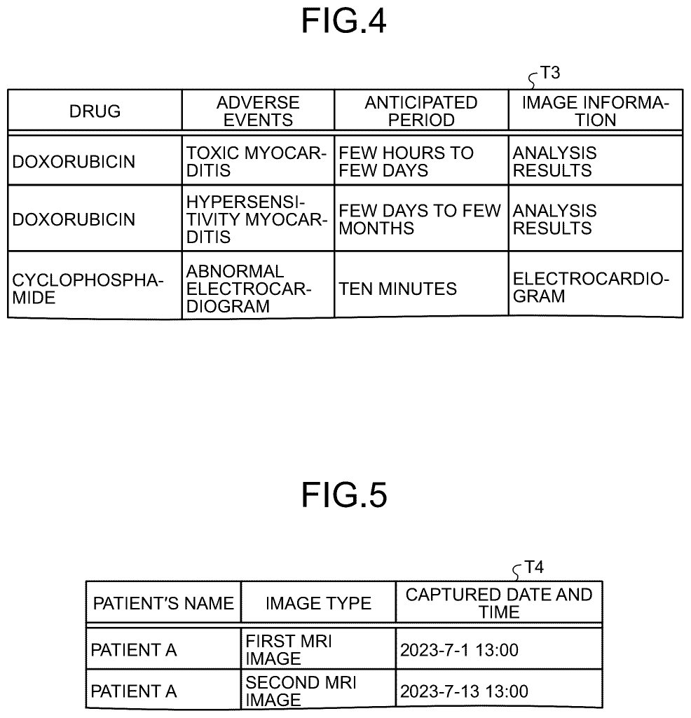

The anticipated period information to be aligned by the aligning function 154 will now be described with reference to FIG. 4. FIG. 4 is a table illustrating an example of detailed drug information according to the embodiment. As illustrated in FIG. 4, detailed drug information T3 is information that includes the administration information including drugs, the adverse event information including the adverse events, the anticipated period information including the anticipated period during which the occurrence of the adverse event is anticipated from the date and time when the drug is administered to the patient, and image information associated with the drugs. The image information associated with the drugs includes analysis results analyzed by other systems, and the analysis results may include the original captured image used for the analysis. The image information will be described below.

Returning to FIG. 1, the first identification function 155 identifies the drug related to the occurrence of the adverse event. The first identification function 155 is an example of a first identification unit. Specifically, from the administration information, the first identification function 155 identifies the related drug related to the occurrence of the adverse event. For example, the first identification function 155 identifies the related drug related to the occurrence of the adverse event, on the basis of the administration information extracted by the extraction function 153, and the anticipated period information that is aligned by the aligning function 154, and that includes the anticipated period during which the occurrence of the adverse event is anticipated from the date and time when the drug is administered to the patient.

For example, the first identification function 155 identifies the related drug information including the drug that caused the occurrence of the adverse event, from the administration date and time of the drug associated with the drug that is included in the administration information illustrated in FIG. 3, and the anticipated period of the adverse event associated with the drug that is included in the detailed drug information T3 illustrated in FIG. 4. In the case of FIG. 3 and FIG. 4, the drug identified by the first identification function 155 is identified as doxorubicin. The related drug information includes the drug name and the effects of the drug. However, the drug information is not limited thereto.

The determination function 156 determines whether there is any drug related to the occurrence of the adverse event. The determination function 156 is an example of a determination unit. Specifically, the determination function 156 determines whether there is any drug identified by the first identification function 155. In this example, if there is a drug identified by the first identification function 155, the determination function 156 determines that there is a drug related to the occurrence of the adverse event. On the other hand, if there is no drug identified by the first identification function 155, the determination function 156 determines that there is no drug related to the occurrence of the adverse event.

The second acquisition function 157 acquires image information. The second acquisition function 157 is an example of a second acquisition unit. Specifically, if the determination function 156 determines that there is a drug related to the occurrence of the adverse event, the second acquisition function 157 acquires image information of the patient to whom the drug related to the occurrence of the adverse event determined by the determination function 156 is administered, from the storage circuit 14.

The image information acquired by the second acquisition function 157 will now be described with reference to FIG. 5. FIG. 5 is a table illustrating an example of image information according to the embodiment. As illustrated in FIG. 5, image information T4 is information that includes patient information including the patient's name, image type information including the image type of the patient, and captured date and time information including the captured date and time when the image is captured.

The second acquisition function 157 may also acquire a plurality of pieces of image information for the image information. For example, the pieces of image information are image information of a patient used to compare between the image information of the patient to whom the drug related to the occurrence of the adverse event is administered, and the image information of the patient to whom the drug related to the occurrence of the adverse event is administered. The image information of the patient to be compared are also referred to as comparison image information. For example, the comparison image information is information on the image data of the patient without any adverse event.

If there is a drug that caused the occurrence of the adverse event, the second identification function 158 identifies the related image information related to the drug. The second identification function 158 is an example of a second identification unit. Specifically, the second identification function 158 identifies the related image information on the related drug, if there is a related drug. For example, the second identification function 158 identifies the related image information that includes the captured image data captured within the anticipated period and that is acquired by the second acquisition function 157, on the basis of the anticipated period included in the anticipated period information aligned by the aligning function 154.

In the case of FIG. 4 and FIG. 5, the captured date and time illustrated in FIG. 5 that is included in the anticipated period illustrated in FIG. 4 is “2023-7-13 13:00”. Hence, the related image information identified by the second identification function 158 is identified as a second magnetic resonance imaging (MRI) image.

The third acquisition function 159 acquires the latest image information. The third acquisition function 159 is an example of a third acquisition unit. Specifically, if the determination function 156 determines that there is no drug related to the occurrence of the adverse event, the third acquisition function 159 acquires the latest image information of the patient with an adverse event, from the storage circuit 14.

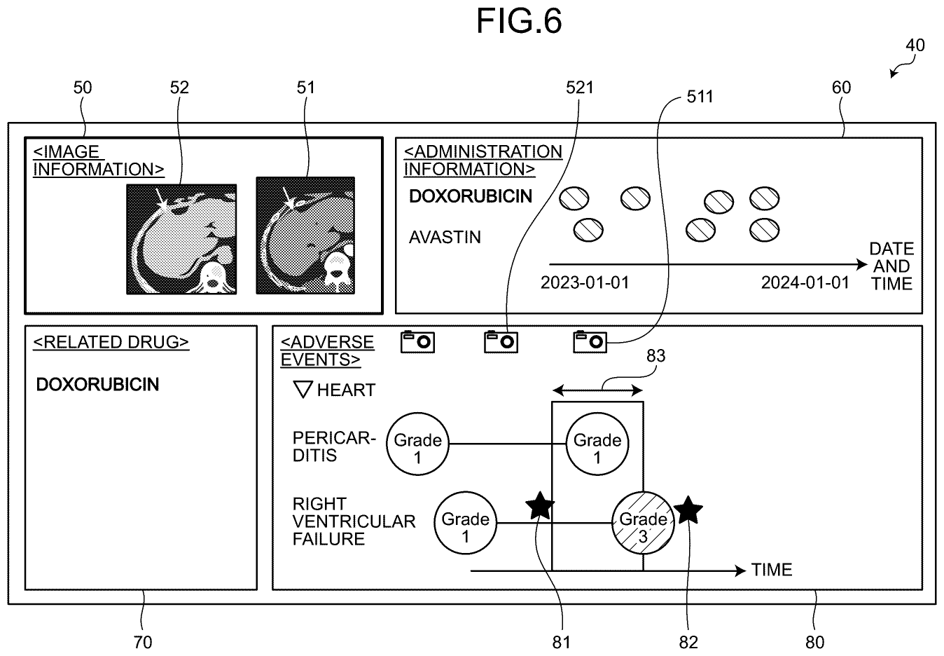

The display control function 160 displays output information. The display control function 160 is an example of a display control unit. Specifically, the display control function 160 displays output information on the display 13. For example, the display control function 160 displays output information that includes the adverse event information, the administration information, drug information on the drug, and the related image information, on the display 13. The output information will now be described with reference to FIG. 6.

FIG. 6 is a diagram for explaining an example of output information according to the embodiment. To specifically explain the present embodiment, the output information illustrated in FIG. 6 is described as information used when a patient develops toxic myocarditis after the administration of doxorubicin, and when the patient's adverse event is diagnosed by a physician.

Output information 40 illustrated in FIG. 6 includes image information 50, administration information 60, related drug information 70, and adverse event information 80. The image information 50 is the image information acquired by the second acquisition function 157. The image information 50 illustrated in FIG. 6 indicates image data 51 included in the related image information, and image data 52 included in the comparison image information. The administration information illustrated in FIG. 6 is the administration information extracted by the extraction function 153. In the administration information 60 illustrated in FIG. 6, doxorubicin and Avastin administered to the patient are indicated in dots such that the date and time of administration can be recognized for each drug.

The related drug information 70 is the related drug information identified by the first identification function 155. The related drug information 70 illustrated in FIG. 6 indicates the drug name of doxorubicin administered to the patient. The adverse event information 80 is the adverse event information and the severity information acquired by the first acquisition function 152. The adverse event information illustrated in FIG. 6 indicates the heart that is a site where the adverse event has occurred in the patient, a mark 81 and a mark 82 that indicate the date and time when the drug is administered to the patient, the name of the adverse event, the CTCAE severity score for the adverse event, and an anticipated period 83.

Moreover, the adverse event information 80 in the output information 40 may also include a mark 511 and a mark 521 that indicate the date and time when the image data 51 and the image data 52 included in the image information 50 are captured. In this manner, for example, in the output information output by the medical information processing device 10, various information on the patient with an adverse event, such as drugs administered to the patient, adverse events, and captured images that indicate the results of cardiac function tests are associated with each other. Hence, it is possible to improve the diagnostic efficiency of physicians, compared to the conventional one.

Moreover, if the determination function 156 determines that there is no drug related to the occurrence of the adverse event, the display control function 160 displays “No related drug”. Then, if the determination function 156 determines that there is no drug related to the occurrence of the adverse event, the display control function 160 displays the latest image information of the patient with an adverse event, acquired by the third acquisition function 159 from the storage circuit 14.

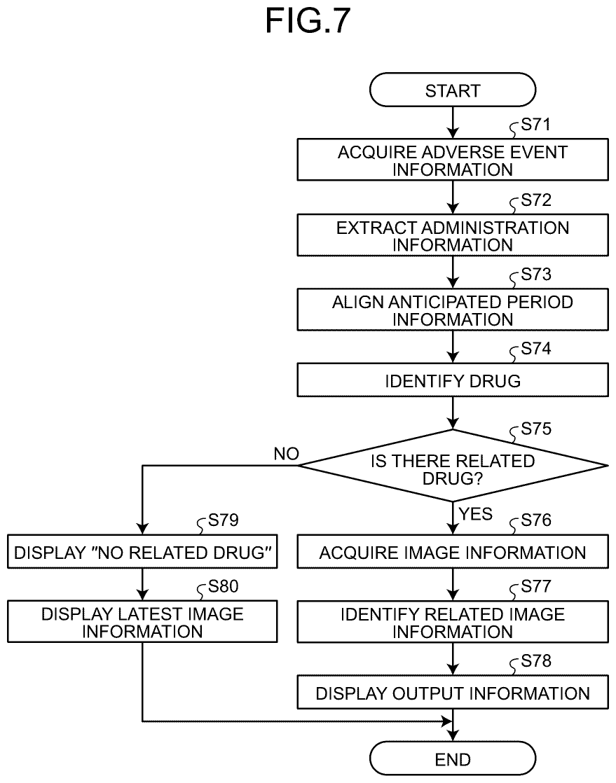

FIG. 7 is a flowchart illustrating an example of a process of the medical information processing device 10 according to the embodiment. It is assumed that the present process is started when a patient develops an adverse event, and after the adverse event information that indicates the adverse event occurred in the patient is registered in the storage circuit 14 by an operator.

First, the first acquisition function 152 acquires the adverse event information from the storage circuit 14 (step S71). Subsequently, the extraction function 153 extracts the administration information administered to the patient, on the basis of the patient information included in the adverse event information acquired by the first acquisition function 152 (step S72).

Subsequently, on the basis of the administration information extracted by the extraction function 153, the aligning function 154 aligns the anticipated period information linked to the drug that is included in the drug information from the detailed drug information stored in the storage circuit 14 (step S73). Subsequently, the first identification function 155 identifies the drug that caused the occurrence of the adverse event, on the basis of the administration information extracted by the extraction function 153 and the anticipated period information aligned by the aligning function 154 (step S74).

The determination function 156 determines whether there is any drug related to the occurrence of the adverse event (step S75). In this process, if there is no drug identified by the first identification function 155, the determination function 156 determines that there is no drug related to the occurrence of the adverse event (No at step S75). Then, the process proceeds to step S79. On the other hand, if there is a drug identified by the first identification function 155, the determination function 156 determines that there is a drug related to the occurrence of the adverse event (Yes at step S75). Then, the process proceeds to step S76.

At step S76, the second acquisition function 157 acquires image information of the patient to whom the drug related to the occurrence of the adverse event determined by the determination function 156 is administered, from the storage circuit 14 (step S76). Subsequently, the second identification function 158 identifies the related image information related to the occurrence of the adverse event that is acquired by the second acquisition function 157 and that is captured within the anticipated period, on the basis of the anticipated period included in the anticipated period information aligned by the aligning function 154 (step S77).

Subsequently, the display control function 160 displays the output information (step S78). At step S79, if the determination function 156 determines that there is no drug related to the occurrence of the adverse event, the display control function 160 displays “No related drug” (step S79). Then, if the determination function 156 determines that there is no drug related to the occurrence of the adverse event, the display control function 160 displays the latest image information of the patient with an adverse event, acquired by the third acquisition function 159 from the storage circuit 14 (step S80). When the present process is finished, the process performed by the processing circuit 15 of the medical information processing device 10 is finished.

As described above, according to the embodiment, the medical information processing device 10 acquires the adverse event information that indicates the adverse event occurred in a patient, extracts the administration information that indicates the drugs administered to the patient, identifies the related drug related to the occurrence of the adverse event from the administration information, identifies the related image information on the related drug, if there is a related drug, and displays the output information that includes the adverse event information, the administration information, the drug information on the drug, and the related image information.

In this manner, for example, when physicians diagnoses a patient with an adverse event, in the output information output by the medical information processing device 10, various information on the patient with an adverse event, such as drugs administered to the patient, adverse events, and captured images that indicate the results of cardiac function tests are associated with each other. Hence, it is possible to improve the diagnostic efficiency of physicians, compared to the conventional method.

Moreover, the medical information processing device 10 identifies the related drug on the basis of the administration information, and the anticipated period information that includes the anticipated period during which the occurrence of the adverse event is anticipated from the date and time when the drug is administered to the patient. Furthermore, the medical information processing device 10 identifies the related image information that includes the captured image data captured within the anticipated period, on the basis of the anticipated period.

In this manner, for example, the medical information processing device 10 identifies the related drug related to the adverse event and the captured image data, from the anticipated period during which the occurrence of the adverse event is anticipated from the date and time when the drug is administered to the patient. Hence, it is possible to improve the diagnostic efficiency of physicians, compared to the conventional method.

Still furthermore, the adverse event information includes severity information that indicates the severity of the adverse event. In this manner, physicians can assess the severity of the adverse event. Hence, it is possible to improve the diagnostic efficiency of physicians, compared to the conventional method.

The embodiment described above may be modified and implemented as appropriate, by changing a part of the configuration or functions of the medical information processing device 10. Therefore, in the following, some modifications according to the embodiment described above will be described as other embodiments. In the following, points different from those in the embodiment described above will be mainly described, and detailed description of points common to the contents already described will be omitted. Moreover, the modifications described below may be implemented individually or in combination as appropriate.

### First Modification

For example, after assessing the related drug in the related drug information 70 included in the output information 40, a physician may identify an alternative drug used as a substitute for the related drug, and consider to administer the identified alternative drug to the patient. Therefore, a mode for identifying the alternative drug will be described in the medical information processing device 10 according to a first modification.

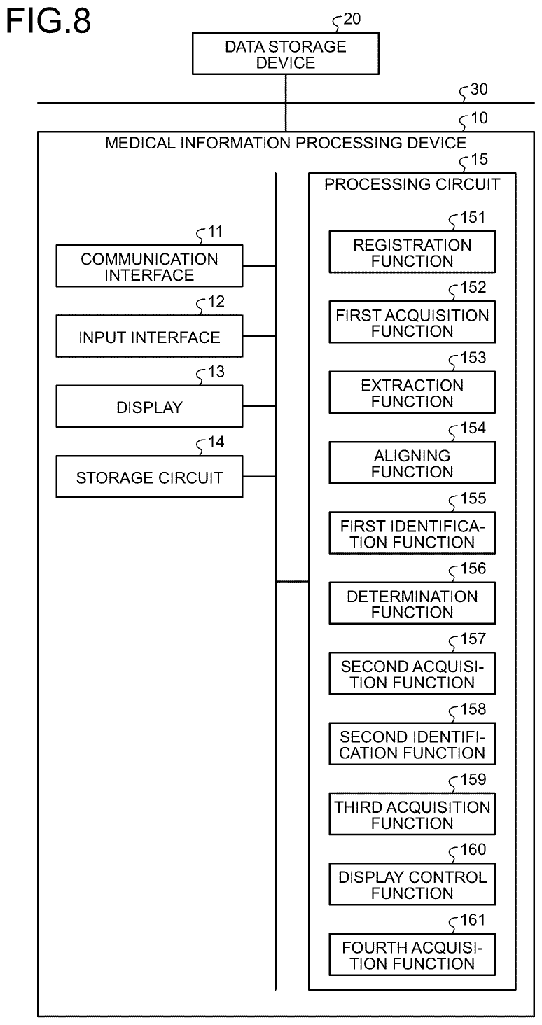

FIG. 8 is a block diagram illustrating the medical information processing device 10 according to the first modification. As illustrated in FIG. 8, the processing circuit 15 of the medical information processing device 10 according to the first modification includes the registration function 151, the first acquisition function 152, the extraction function 153, the aligning function 154, the first identification function 155, the determination function 156, the second acquisition function 157, the second identification function 158, the third acquisition function 159, the display control function 160, and a fourth acquisition function 161.

The fourth acquisition function 161 acquires input operation information. The fourth acquisition function 161 is an example of a fourth acquisition unit. Specifically, by cooperatively working with the input interface 12 operated by an operator, the fourth acquisition function 161 acquires the input operation information by operating the output information 40. For example, the fourth acquisition function 161 acquires the input operation information that is obtained by pressing the severity information (for example, the CTCAE severity score and the like) in the adverse event information 80 included in the output information 40. However, the input operation information is not limited thereto. For example, the input operation information may also be obtained by operating the drug name of the related drug in the related drug information 70.

On the basis of the input operation information acquired by the fourth acquisition function 161, the first identification function 155 identifies the alternative drug used as a substitute for the related drug that caused the adverse event corresponding to the input operation information. For example, the first identification function 155 acquires information on the drug from the data storage device 20, and on the basis of the input operation information acquired by the fourth acquisition function 161, identifies the alternative drug to be used as a substitute for the related drug that caused the adverse event corresponding to the input operation information. The information on the drug may also be stored in the storage circuit 14.

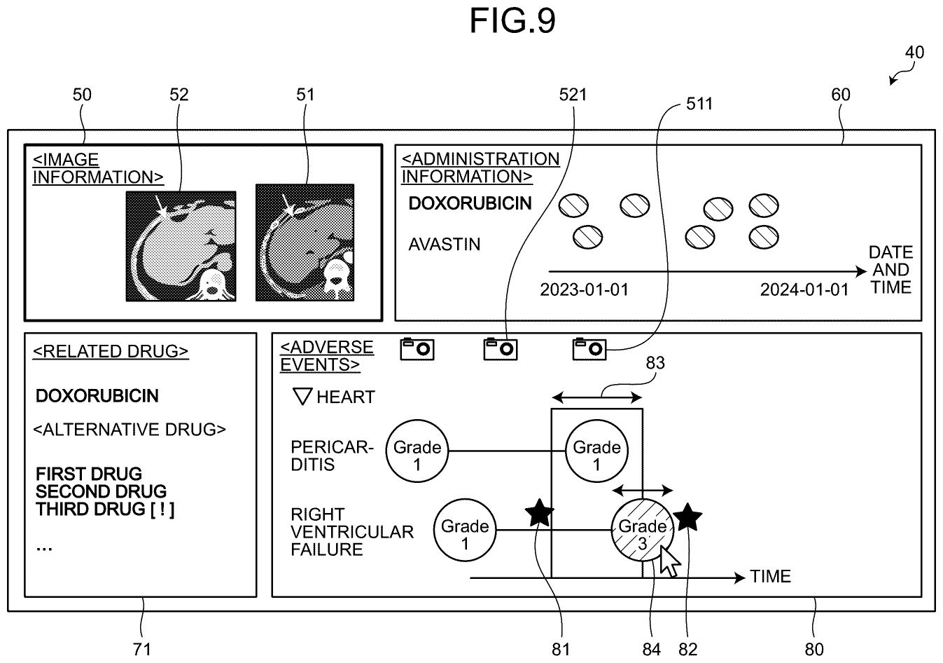

The display control function 160 updates the related drug information 70 included in the output information 40. Specifically, the display control function 160 adds the drug name according to the alternative drug identified by the first identification function 155 to the related drug information 70, and updates to the related drug information 71. Then, the display control function 160 displays the output information 40 that includes the updated related drug information on the display 13. The updated output information 40 will now be described with reference to FIG. 9.

FIG. 9 is a diagram for explaining an example of the output information 40 according to the first modification. To specifically explain the present modification, it is assumed that the output information 40 illustrated in FIG. 9 is operated, after the output information 40 illustrated in FIG. 6 is checked by a physician.

For example, after checking the output information 40 illustrated in FIG. 6, the physician may consider an alternative drug used as a substitute for doxorubicin that is the related drug administered to the patient, as illustrated in the related drug information 70. In this case, the physician presses a CTCAE severity score 84 illustrated in FIG. 9.

Then, by cooperatively working with the input interface 12 operated by the physician, the fourth acquisition function 161 acquires the input operation information that is obtained by pressing the CTCAE severity score 84. Subsequently, the first identification function 155 acquires information on the drug from the data storage device 20, and identifies the alternative drug used as a substitute for doxorubicin that is the related drug corresponding to the CTCAE severity score 84.

Subsequently, the display control function 160 adds the drug name according to the alternative drug to doxorubicin that is identified by the first identification function 155 to the related drug information 70, updates the related drug information 70 to the related drug information 71, and displays the output information 40 that includes the updated related drug information 71 on the display 13.

The updated related drug information 71 illustrated in FIG. 9 indicates the drug name of doxorubicin that is the related drug administered to the patient, and a first drug, a second drug, and a third drug [!] that are the alternative drugs to doxorubicin. The third drug needs to be careful when used in combination with Avastin that is administered to the patient. Hence, [!] is attached as a warning sign. In this manner, for example, by checking the output information 40 in which the related drug information 71 is updated, the physician can consider the alternative drug to the related drug. Hence, it is possible to improve the diagnostic efficiency of physicians, compared to the conventional method.

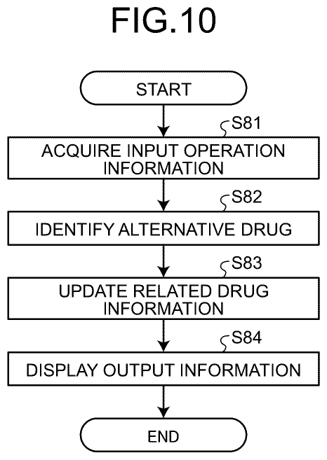

FIG. 10 is a flowchart illustrating an example of a process of the medical information processing device 10 according to the first modification. It is assumed that the present process is started after the output information 40 is operated, after the output information 40 illustrated in FIG. 6 is checked by a physician.

The fourth acquisition function 161 acquires the input operation information that is obtained by pressing the severity information (for example, the CTCAE severity score and the like) in the adverse event information 80 included in the output information 40 (step S81). Subsequently, on the basis of the input operation information acquired by the fourth acquisition function 161, the first identification function 155 identifies the alternative drug used as a substitute for the related drug that caused the adverse event corresponding to the input operation information (step S82).

Subsequently, the display control function 160 adds the drug name according to the alternative drug identified by the first identification function 155 to the related drug information 70, and updates the related drug information 70 to the related drug information 71 (step S83). Then, the display control function 160 displays the output information 40 that includes the updated related drug information 71 on the display 13 (step S84).

As described above, according to the first modification, the medical information processing device 10 acquires the input operation information that is obtained by pressing the severity information in the adverse event information 80 included in the output information 40, and on the basis of the input operation information, identifies the alternative drug used as a substitute for the related drug that caused the adverse event corresponding to the input operation information. Then, the medical information processing device 10 adds the drug name according to the identified alternative drug to the related drug information 70, updates the related drug information 70 to the related drug information 71, and displays the output information 40 that includes the updated related drug information 71 on the display 13.

In this manner, for example, by checking the output information 40 in which the related drug information 71 is updated, the physician can consider the alternative drug to the related drug. Hence, it is possible to improve the diagnostic efficiency of physicians, compared to the conventional method.

### Second Modification

For example, a physician may check the anticipated period for each severity information in the adverse event information 80 included in the output information 40. Therefore, a mode for displaying the anticipated period for each severity information will be described in the medical information processing device 10 according to a second modification.

For example, the fourth acquisition function 161 acquires the input operation information that is obtained by pressing the severity information (for example, the CTCAE severity score and the like) in the adverse event information 80 included in the output information 40. For example, the first identification function 155 identifies the drug corresponding to the severity information included in the input operation information acquired by the fourth acquisition function 161, from the administration information T2 stored in the storage circuit 14.

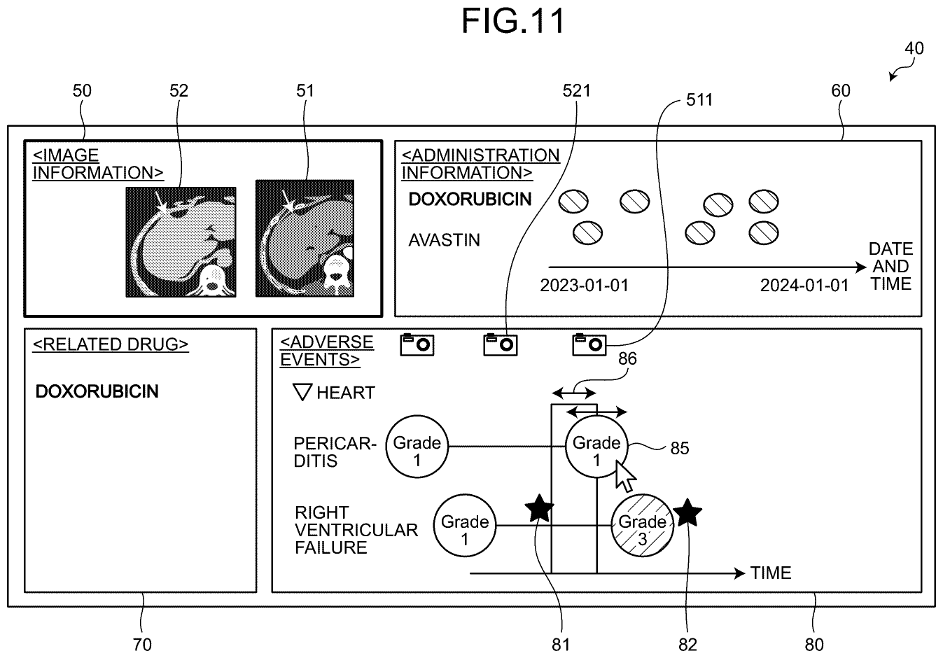

The display control function 160 updates the anticipated period included in the output information 40. Specifically, the display control function 160 updates the anticipated period displayed in the output information 40, to the anticipated period corresponding to the drug identified by the first identification function 155. Then, the display control function 160 displays the output information 40 that includes the updated anticipated period on the display 13. The updated output information 40 will now be described with reference to FIG. 11.

FIG. 11 is a diagram for explaining an example of the output information 40 according to the second modification. To specifically explain the present modification, it is assumed that the output information 40 illustrated in FIG. 10 is operated, after the output information 40 illustrated in FIG. 6 is checked by a physician.

For example, after checking the output information 40 illustrated in FIG. 6, the physician may wish to check the anticipated period for each severity information in the adverse event information 80. In this case, the physician presses a CTCAE severity score 85 illustrated in FIG. 9.

Then, by cooperatively working with the input interface 12 operated by the physician, the fourth acquisition function 161 acquires the input operation information that is obtained by pressing the CTCAE severity score 85. Subsequently, the first identification function 155 identifies doxorubicin corresponding to the CTCAE severity score 85, included in the input operation information acquired by the fourth acquisition function 161, from the administration information T2 stored in the storage circuit 14.

Subsequently, the display control function 160 updates the anticipated period 83 displayed in the output information 40, to an anticipated period 86 corresponding to doxorubicin identified by the first identification function 155, and displays the output information 40 that includes the updated anticipated period 86 on the display 13.

In this manner, for example, by checking the output information 40 in which the anticipated period 86 is updated, the physician can check the anticipated period for each severity information in the adverse event information 80. Hence, it is possible to improve the diagnostic efficiency of physicians, compared to the conventional method.

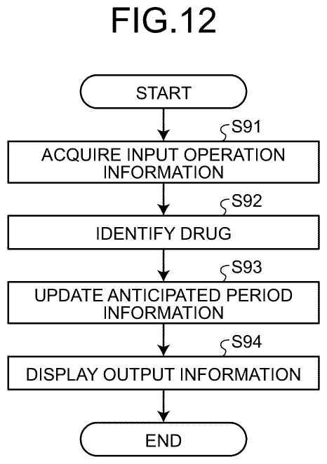

FIG. 12 is a flowchart illustrating an example of a process of the medical information processing device 10 according to the second modification. It is assumed that the present process is started after the output information 40 is operated, after the output information 40 illustrated in FIG. 6 is checked by a physician.

The fourth acquisition function 161 acquires the input operation information that is obtained by pressing the severity information (for example, the CTCAE severity score and the like) in the adverse event information 80 included in the output information 40 (step S91). Subsequently, the first identification function 155 identifies the drug corresponding to the severity information included in the input operation information acquired by the fourth acquisition function 161, from the administration information T2 stored in the storage circuit 14 (step S92).

Subsequently, the display control function 160 updates the anticipated period 83 displayed in the output information 40, to the anticipated period 86 corresponding to the drug identified by the first identification function 155 (step S93). Then, the display control function 160 displays the output information 40 that includes the updated anticipated period 86 on the display 13 (step S94).

As described above, according to the second modification, the medical information processing device 10 acquires the input operation information that is obtained by pressing the severity information in the adverse event information 80 included in the output information 40, and identifies the drug corresponding to the severity information included in the input operation information. Then, the medical information processing device 10 updates the anticipated period 83 displayed in the output information 40, to the anticipated period 86 corresponding to the drug identified by the first identification function 155, and displays the output information 40 that includes the updated anticipated period 86 on the display 13.

In this manner, for example, by checking the output information 40 in which the anticipated period 86 is updated, the physician can check the anticipated period for each severity information in the adverse event information 80. Hence, it is possible to improve the diagnostic efficiency of physicians, compared to the conventional method.

### Third Modification

In the present embodiment, the adverse events are cardiotoxic symptoms caused by anticancer drugs. However, it is not limited thereto. The adverse events are not only caused by drugs such as anticancer drugs, and the medical information processing device 10 is applicable to any drug that causes an adverse event.

In the embodiments and modifications described above, the registration unit, the first acquisition unit, the extraction unit, the aligning unit, the first identification unit, the determination unit, the second acquisition unit, the second identification unit, the third acquisition unit, the display control unit, and the fourth acquisition unit in the present specification are each implemented by the registration function, the first acquisition function, the extraction function, the aligning function, the first identification function, the determination function, the second acquisition function, the second identification function, the third acquisition function, the display control function, and the fourth acquisition function of the processing circuit. However, the embodiments are not limited thereto. For example, in addition to implementing the registration unit, the first acquisition unit, the extraction unit, the aligning unit, the first identification unit, the determination unit, the second acquisition unit, the second identification unit, the third acquisition unit, the display control unit, and the fourth acquisition unit in the present specification by the registration function, the first acquisition function, the extraction function, the aligning function, the first identification function, the determination function, the second acquisition function, the second identification function, the third acquisition function, the display control function, and the fourth acquisition function described in the embodiments, the same functions may also be implemented using hardware only, software only, or a combination of hardware and software.

Moreover, for example, the term “processor” used in the above embodiments refers to a central processing unit (CPU), a graphics processing unit (GPU), or a circuit such as an application specific integrated circuit (ASIC) and a programmable logic device (for example, a simple programmable logic device (SPLD), a complex programmable logic device (CPLD), and a field programmable gate array (FPGA)).

In this example, instead of storing a computer program in the storage circuit, a computer program may also be directly embedded in a circuit of the processor. In this case, the processor implements the functions by reading and executing a computer program embedded in the circuit. Moreover, each processor in the present embodiments is not limited to being configured as a single circuit for each processor, but may also be configured as a single processor by combining multiple independent circuits to realize the functions.

In this example, a medical information processing program to be executed by the processor is provided by being incorporated in the read only memory (ROM), the storage circuit, and the like in advance. The medical information processing program may also be provided by being recorded on a computer readable non-transitory storage medium such as a compact disk ROM (CD-ROM), a flexible disk (FD), a CD-Recordable (CD-R), and a digital versatile disk (DVD) in a file format that can be installed or executed on these devices.

Moreover, the medical information processing program may also be stored on a computer connected to a network such as the Internet, and provided or distributed by being downloaded via a network. For example, the medical information processing program is configured by a module including the processing functions described above. As an actual hardware, the CPU reads and executes the medical image processing program from the storage medium such as the ROM, and each module is loaded onto the main storage device, and generated on the main storage device.

Moreover, in the embodiments and modifications described above, each component of each device illustrated in the drawings is functionally conceptual, and need not necessarily be physically configured as illustrated in the drawings. That is, a specific mode of dispersion or integration of each device is not limited to those illustrated in the drawings, and all or part thereof may be configured in a functionally or physically dispersed or integrated manner in any units according to various loads and usage conditions. Furthermore, all or any part of the processing functions performed by the devices can be implemented by a CPU and a computer program analyzed and executed by the CPU, or can be implemented as hardware by wired logic.

Of the processes described in the above embodiments and modifications, all or part of the processes described as being automatically performed may be manually performed, or all or part of the processes described as being manually performed may be automatically performed with a known method. In addition, the processing procedures, control procedures, specific names, and information including various kinds of data and parameters illustrated in the above document and drawings can be optionally changed unless otherwise specified.

According to at least one of the embodiments described above, the diagnostic efficiency can be improved compared to the conventional method.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing device, comprising:
a processor configured to
acquire adverse event information that indicates an adverse event occurred in a patient,
extract administration information that indicates a drug administered to the patient,
identify a related drug related to occurrence of the adverse event, from the administration information,
identify related image information on the related drug, when there is the related drug, and
display output information that includes the adverse event information, the administration information, drug information on the drug, and the related image information.

2. The medical information processing device according to claim 1, wherein the processor identifies the related drug, based on the administration information, and anticipated period information that includes an anticipated period during which occurrence of the adverse event is anticipated from date and time when the drug is administered to the patient.

3. The medical information processing device according to claim 2, wherein the processor identifies the related image information that includes captured image data captured within the anticipated period, based on the anticipated period.

4. The medical information processing device according to claim 1, wherein the adverse event information includes severity information that indicates severity of the adverse event.

5. A medical information processing method, comprising:
acquiring adverse event information that indicates an adverse event occurred in a patient;
extracting administration information that indicates a drug administered to the patient;
identifying a related drug related to occurrence of the adverse event, from the administration information;
identifying related image information on the related drug, when there is the related drug; and
displaying output information that includes the adverse event information, the administration information, drug information on the drug, and the related image information.

6. A non-transitory computer readable medium including programmed instructions, wherein the instructions, when executed by a computer, cause the computer to execute:
acquiring adverse event information that indicates an adverse event occurred in a patient;
extracting administration information that indicates a drug administered to the patient;
identifying a related drug related to occurrence of the adverse event, from the administration information;
identifying related image information on the related drug, when there is the related drug; and
displaying output information that includes the adverse event information, the administration information, drug information on the drug, and the related image information.
